(19) United States
(12) Patent Application Publication
(10) Pub. No.: US 2002/0128560 A1
(43) Pub. Date: Sep. 12, 2002

(54) Surgical flow restrictor and filter

(76) Inventor: Alex Urich, Mission Viejo, CA (US)

Correspondence Address: IRELL & MANELLA LLP, 840 NEWPORT CENTER DRIVE, SUITE 400, NEWPORT BEACH, CA, 92660, US

(21) Appl. No.: 09/922,473

(22) Filed: Aug. 3, 2001

Related U.S. Application Data
(60) Provisional application No. 60/274,451, filed on Mar. 9, 2001.

(51) Int. Cl. A61M 1/00; A61M 5/00

(52) U.S. Cl. 600/500; 604/252; 604/256; 604/290; 604/35; 604/246

(57) ABSTRACT

A medical system that may include a filter/flow restrictor which limits the pressure drop within an aspiration system. The restrictor may include a flow restrictor that is coupled to a filter housing. The flow restrictor restricts the pressure drop within the aspiration system. The housing may contain a filter which prevents occlusion of the flow restrictor. When used in an opthtalmic procedure the flow restrictor may limit the minimum pressure within a cornea and prevent corneal collapse.

### Cross-Reference to Related Application

The present application claims the benefit of U.S. Provisional Application No. 60/274,451, filed Mar. 9, 2001.

### BACKGROUND OF THE INVENTION

1. Field of the Invention

The present application relates to a filter and flow restrictor for a medical aspiration system.

2. Background

The lens of a human eye may develop a cataracteous condition which affects a patients vision. Cataracteous lenses are sometimes removed and replaced in a procedure commonly referred to as phacoemulsification. Phaco procedures are typically performed with an ultrasonically driven handpiece which is used to break the lens. The broken lens is removed through an aspiration line that is coupled to the handpiece.

The handpiece has a tip which is inserted through an incision in the cornea. The handpiece typically contains a number of ultrasonic transducers that convert electrical power into a mechanical oscillating movement of the tip. The distal end of the tip has an opening that is in fluid communication with the aspiration line. The distal end of the tip also has a sleeve which has an opening in fluid communication with an irrigation line. The irrigation line is typically connected to a bottle that can provide irrigation fluid to the surgical site.

The oscillating movement of the tip will break the lens into small pieces. The lens pieces and irrigation fluid are drawn into the aspiration line through the opening of the tip. When performing a phaco procedure it is essential to maintain a positive pressure within the anterior chamber of the eye. A negative pressure may cause the cornea to collapse. To maintain a positive chamber pressure the system is configured to provide a flowrate through the irrigation tube that is greater than the flowrate through the aspiration tube.

It has been found that the aspiration tube may become occluded during a procedure. The occlusion will increase the vacuum pressure within the aspiration line. When the occlusion is cleared the anterior chamber may be instantaneous exposed to a high vacuum pressure. The vacuum pressure may cause the cornea to collapse.

### BRIEF SUMMARY OF THE INVENTION

A flow restrictor that includes a filter located within a filter housing and a flow restrictor coupled to the housing.

### BRIEF DESCRIPTION OF THE DRAWINGS

FIG. 1 is an illustration of a medical system with a filter/flow restrictor;

FIG. 2 is a cross-sectional view of an aspiration tube assembly of the system;

FIG. 3 is a graph showing a flowrate versus a line vacuum pressure for, an aspiration system with a flow restrictor, and an aspiration system without a flow restrictor;

FIG. 4 is a graph showing the line vacuum pressure versus the pressure within a cornea.

### DETAILED DESCRIPTION

Disclosed is a medical system that may include a filter/flow restrictor which limits the pressure drop within an aspiration system. The restrictor may include a flow restrictor that is coupled to a filter housing. The flow restrictor restricts the pressure drop within the aspiration system. The housing may contain a filter which prevents occlusion of the flow restrictor. When used in an opthtalmic procedure the flow restrictor may limit the minimum pressure within a cornea and prevent corneal collapse.

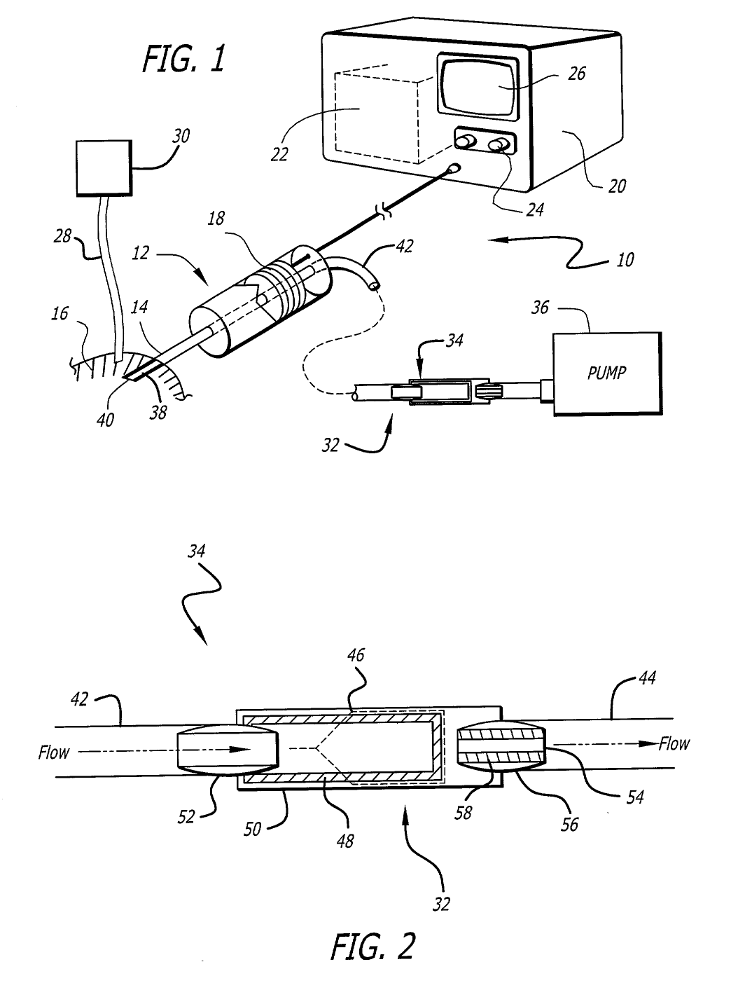

Referring to the drawings more particularly by reference numbers, FIG. 1 shows an embodiment of a medical system 10. The system 10 may include an ultrasonically driven handpiece which has a tip 14 that can be inserted into a cornea 16. The tip 14 may also be referred to as a cutting element. The handpiece 12 may include one or more ultrasonic transducers 18 that convert electrical power into mechanical movement of the tip 14. The handpiece 12 is typically held by-a surgeon who performs a surgical procedure with the system 10. By way of example, the system 10 can be used to perform a phacoemulsification procedure to break and aspirate a lens of the cornea 16.

The handpiece 12 may be connected to a console 20 of the system 10. The console 20 may contain a control circuit 22 that provides a driving signal to the transducers 18. The console 20 may have input knobs or buttons 24 that allow the surgeon to vary different parameters of the system 10. The console 20 may also have a readout display 26 that provides an indication of the power level, etc. of the system 10.

The system 10 may include an irrigation tube 28 that is connected to an irrigation bottle 30. The irrigation tube 28 can be inserted into the cornea 16. The irrigation bottle 30 may contain an irrigation fluid that flows into the cornea 16 through the irrigation tube 28.

The medical system 10 may further have an aspiration system 32 that aspirates the irrigation fluid and broken lens out of the cornea 16. The aspiration system 32 may include an aspiration tube assembly 34 that is connected to the handpiece 12 and a vacuum pump 36. The aspiration tube assembly 34 is in fluid communication with an inner channel 38 and an opening 40 of the tip 14. The vacuum pump 36 creates a negative pressure within the aspiration tube assembly 34 to induce a flow of irrigation fluid and emulsified tissue out of the cornea 16. The pump 36 is configured so that the flowrate through the irrigation tube 28 is slightly greater than the flowrate through the aspiration tube assembly 34.

As shown in FIG. 2, the aspiration tube assembly 34 may include an input tube 42 and an output tube 44 that are coupled to a flow restrictor 46. The flow restrictor 46 includes a filter 48 that is located within a filter housing 50. The filter 48 may be a mesh type device that filters out particles and contaminates in the fluid flowing through the housing 50. It is preferable to have the filter 48 pressed into the housing 50 so that there is not a space between the outer filter surface and the inner housing surface. Press fitting the filter 48 into the filter housing 50 prevents air pockets from forming in the housing 50 and allows for proper pump priming of the restrictor 46.

The input tube 42 may include an input luer 52 that couples the tube 42 to the housing 50. The input luer 52 is preferably inserted into the filter 48 to prevent fluid from becoming trapped in the entrance of the housing 50. It is desirable to provide a filter 48 that has an outer diameter that is no greater than twice the diameter of the input luer 52. It has been found that such a ratio will insure that the restrictor 46 will be properly primed during aspiration. By way of example, the filter 48 may have a diameter of approximately 0.1 inch.

The restrictor 46 may include a flow restrictor 54 coupled to the filter housing 50. The flow restrictor 54 limits the variance in flowrate of the irrigation fluid and the corresponding pressure drop across the aspiration system 32.

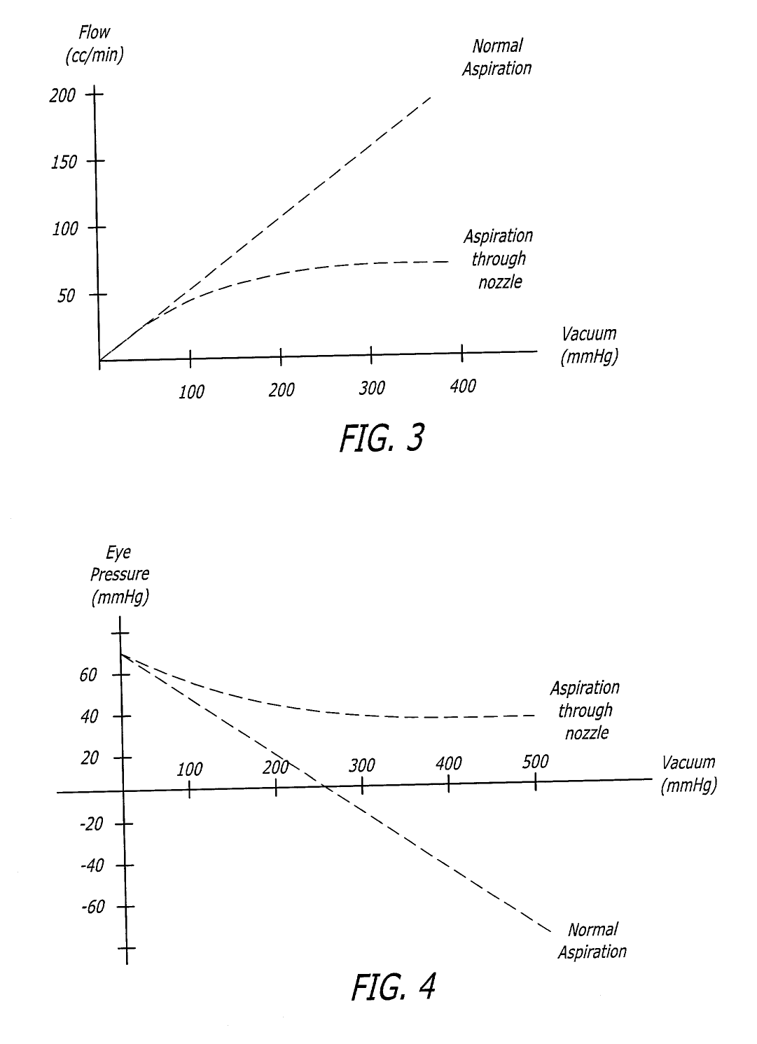

FIG. 3 shows the vacuum pressure versus flowrate for an aspiration system with a flow restrictor, and a system without a restrictor. The vacuum pressure depicted is downstream from the restrictor. As can be seen a non-restrictor system will have a linear increase in flow with an increase in vacuum pressure. In contrast to the prior art, the flow restrictor 54 of the restrictor creates a non-linear increase in the flowrate that levels off after the vacuum pressure exceeds a certain level. The restriction of fluid flow limits the pressure drop in the cornea as shown in FIG. 4 to prevent negative chamber pressure as indicated by the graph. By utilizing a flow restrictor, the aspiration system can prevent a negative pressure in the cornea anterior chamber that will cause a corneal collapse.

The flow restrictor 54 may be integrated into an output luer 56 that is attached to the filter housing 50 and pressed into the output tube 44. The output luer 56 may have a detachable scaling insert 58 that defines the diameter of the flow restrictor 54. The diameter of the flow restrictor 54 may be varied by inserting a different scaling insert 58. The diameter of the flow restrictor 54 defines the upper limit of the flowrate in the aspiration system (see FIG. 3). The operator can vary the upper flowrate by inserting a different insert 58 and/or luer 56 into the tube assembly 34. By way of example, the flow restrictor 54 may have a diameter between 0.1 and 1.0 millimeters and a length at least 25.4 millimeters. This range will provide the nozzle effect shown in FIGS. 3 and 4 when fitted with convention asporation tubing found in medical systems. For example, conventional aspiration tubing diameters may range from 1.52 to 2.54 millimeters.

The filter 48 and housing 50 are preferably constructed from a disposable material. To insure that the restrictor 46 has capacity for an entire procedure it is preferable to provide a filter 48 which has a volume that can capture two cataract lenses. By way of example, the filter 48 may have a volume of approximately 1 cubic centimeter.

Referring to FIGS. 1 and 2, in operation, an operator attaches a restrictor 46 to the input 42 and output 44 tubes. A medical procedure is then performed on a cornea 16. The aspiration system 32 may develop an occlusion which increases the downstream vacuum pressure of the system. The flow restrictor 54 limits the flowrate from the cornea 16 to prevent corneal collapse when the occlusion is cleared from the system. The filter 48 prevents the flow restrictor 54 from being occluded to allow for normal fluid flow during a procedure. After the procedure is completed the flow restrictor 46 is replaced with another restrictor unit.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A flow restrictor for a medical aspiration system, comprising:
a filter housing;
a flow restrictor coupled to said filter housing; and,
a filter located within said filter housing.

2. The flow restrictor of claim 1, wherein said flow restrictor has a diameter between 0.1 to 1 millimeters.

3. The flow restrictor of claim 1, wherein said flow restrictor is located within an output luer attached to said filter housing.

4. The flow restrictor of claim 3, wherein said output luer includes a scaling insert.

5. An aspiration tube assembly for a medical system, comprising:
an input tube;
an input luer coupled to said input tube, said input luer having a diameter;
a filter housing coupled to said input luer;
a filter located within said filter housing, said filter having a diameter that is no greater than twice the diameter of said input luer; and,
a flow restrictor coupled to said filter housing.

6. The aspiration tube assembly of claim 5, wherein said input luer is pressed into said filter.

7. The aspiration tube assembly of claim 5, wherein said filter is pressed into said filter housing.

8. The aspiration tube assembly of claim 5, wherein said flow restrictor has a diameter between 0.1 to 1 millimeters.

9. The aspiration tube assembly of claim 5, wherein said flow restrictor is located within an output luer attached to said filter housing.

10. The aspiration tube assembly of claim 9, wherein said output luer includes a scaling insert.

11. An aspiration tube assembly for a medical system, comprising:
an input tube;
an input luer coupled to said input tube;
a filter housing coupled to said input luer;
a filter located within said filter housing and pressed into said input luer; and,
a flow restrictor coupled to said filter housing.

12. The aspiration tube assembly of claim 11, wherein said filter is pressed into said filter housing.

13. The aspiration tube assembly of claim 11, wherein said flow restrictor has a diameter between 0.1 to 1 millimeters.

14. The aspiration tube assembly of claim 11, wherein said flow restrictor is located within an output luer attached to said filter housing.

15. The aspiration tube assembly of claim 14, wherein said output luer includes a scaling insert.

16. A flow restrictor for a medical aspiration system, comprising:
a filter housing;
filter means for filtering a flow of fluid through said filter housing; and,
flow restrictor means for restricting the flow of fluid through said filter housing.

17. The flow restrictor of claim 16, wherein said flow restrictor means includes a flow restrictor with a diameter between 0.1 to 1 millimeters.

18. The flow restrictor of claim 16, wherein said flow restrictor means includes an output luer attached to said filter housing.

19. The flow restrictor of claim 18, wherein said output luer includes a scaling insert.

20. An aspiration tube assembly for a medical system, comprising:
an input tube;
a filter housing coupled to said input tube;
filter means for filtering a flow of fluid through said filter housing;
input means for coupling said input tube to said filter means; and
flow restrictor means for restricting the flow of fluid through said filter housing.

21. The aspiration tube assembly of claim 20, wherein said input means includes an input luer that is pressed into said filter means.

22. The aspiration tube assembly of claim 20, wherein said filter means includes a filter that is pressed into said filter housing.

23. The aspiration tube assembly of claim 20, wherein said flow restrictor means includes a flow restrictor that has a diameter between 0.1 to 1 millimeters.

24. The aspiration tube assembly of claim 20, wherein said flow restrictor means includes an output luer attached to said filter housing.

25. The aspiration tube assembly of claim 24, wherein said output luer includes a scaling insert.

26. A method for aspirating a cornea, comprising:
inducing a flow of fluid out of the cornea;
filtering the fluid; and,
restricting the flow of filtered fluid.

27. The method of claim 26, further comprising attaching a filter and a flow restrictor to an input tube and an output tube.

28. The method of claim 27, further comprising detaching the filter and the flow restrictor from the input tube and the output tube.
